(19) United States
(12) Patent Application Publication
(10) Pub. No.: US 2021/0121508 A1
(43) Pub. Date: Apr. 29, 2021

(54) VAGINITIS PATHOGEN-INHIBITING COMPOSITION, VAGINA-CLEANING COMPOSITION, AND USES THEREOF

(71) Applicant: GLAC BIOTECH CO., LTD., TAINAN CITY (TW)

(72) Inventors: PEI-SHAN HSIEH, Tainan City (TW); CHUNG-WEI KUO, Tainan City (TW); YI-CHUN TSAI, Tainan City (TW); HSIEH-HSUN HO, Tainan City (TW); YI WEI KUO, Tainan City (TW); YU FEN HUANG, Tainan City (TW)

(21) Appl. No.: 16/940,149

(22) Filed: Jul. 27, 2020

(30) Foreign Application Priority Data
Oct. 29, 2019 (CN) 201911038378.X

(51) Int. Cl. A61K 35/747; A61K 35/745; A23L 33/135; A23C 9/123

(52) U.S. Cl. CPC A61K 35/747; A61K 35/745; A61K 45/06; A23C 9/1234; A23L 33/135

(57) ABSTRACT

A vaginitis pathogen-inhibiting composition comprises a fermentate of lactic acid bacteria, which is generated by jointly fermenting a lactic acid bacteria strain and Streptococcus thermophilus in a medium containing at least one of milk, powdered milk, and casein. The lactic acid bacteria strain includes an AP-32 strain of Lactobacillus salivarius subsp. Salicinius; an F-1 strain of Lactobacillus rhamnosus; a GL-165 strain of Lactobacillus rhamnosus; a CP-9 strain of Bifidobacterium animalis subsp. lactis; a Bf-688 strain of Bifidobacterium bifidum; a Bv-889 strain of Bifidobacterium breve; or combinations thereof. The above-mentioned fermentate of the lactic acid bacteria strain can inhibit growth of vaginitis pathogens and may be in form of a food composition, a pharmaceutical composition, or a vagina-cleaning composition.

### BACKGROUND OF THE INVENTION

### 1. Field of the Invention

The preset invention relates to compositions and uses thereof, particularly to a vaginitis pathogen-inhibiting composition, a vagina-cleaning composition, and uses thereof.

### 2. Description of the Prior Art

There have been many researches about the effects of probiotics on health of human bodies. In general, the bacteria strains having special functions to health of human bodies are called functional probiotics. Please refer to Guidelines for the evaluation of probiotics in food; Report of joint FAO/WHO working group on drafting guidelines for the evaluation of probiotics in food; London Ontario, Canada April 30 and May 1, 2002: 1-7. The special functions of probiotics include regulating gastrointestinal functions, regulating immunological functions, and even inhibiting inflammation. It is the specificity of a strain but not the whole species of a probiotic that can contribute special functions to human health. In other words, different strains may have different functions or even opposite functions although they belong to the same species.

Some researches point out that over 50% modern women suffer urinary-tract infection or vaginal infection. Some syndromes of lower genital tracts are usually seen in clinics, such as dysuria, pruritus, and secreta. These syndromes are often attributed to vaginal infections. The vaginal infections of childbearing women normally result from candidiasis, bacterial vaginal diseases, and trichomoniasis. The front two are caused by microorganisms. The vagina is accessible to the external environment and neighbors the anus where many pathogens exist. Therefore, the vagina is very likely to be invaded by pathogens. Thus, the human bodies have a defense system to prevent from overgrowth of pathogens.

The vagina's defense system is mainly based on an acidic environment having a pH value of lower than 4.5. Although the acidic environment is unfavorable to growth of pathogens, it is an ideal environment for acidophil bacilli. The acidophil bacilli may further generate additional acid to maintain a healthy environment. However, the activity of the acidophil bacilli may be decreased by some factors, such as broad-spectrum antibiotics. In such a case, the pH value of the vagina will rise, and pathogens may grow too much and cause infections.

Accordingly, developing products able to inhibit vaginitis pathogens has become a target the manufactures desire to achieve.

### SUMMARY OF THE INVENTION

The present invention provides a vaginitis pathogen-inhibiting composition, which comprises a fermentate generated by jointly fermenting lactic acid bacteria strains and Streptococcus thermophilus in a medium containing at least one of milk, powdered milk, and casein. The fermentate of the lactic acid bacteria strains can inhibit growth of vaginitis pathogens and may be in form of a food composition, a pharmaceutical composition, or a vagina-cleaning composition.

In one embodiment, the vaginitis pathogen-inhibiting composition of the present invention comprises a fermentate of lactic acid bacteria, and an excipient, diluent or carrier. The fermentate of lactic acid bacteria is generated by jointly fermenting an isolated lactic acid bacteria strain and Streptococcus thermophilus in a medium containing at least one of milk, powdered milk, and casein. The lactic acid bacteria strain comprises an AP-32 strain of Lactobacillus salivarius subsp. Salicinius (Deposition Number: CCTCC NO. M2011127); an F-1 strain of Lactobacillus rhamnosus (Deposition Number: CCTCC NO. M2011124); a GL-165 strain of Lactobacillus rhamnosus (Deposition Number: CCTCC NO. M2014591); a CP-9 strain of Bifidobacterium animalis subsp. lactis (Deposition Number: CCTCC NO. M2014588); a Bf-688 strain of Bifidobacterium bifidum (Deposition Number: CGMCC NO. 17953); a Bv-889 strain of Bifidobacterium breve (Deposition Number: CGMCC NO. 16145) or combinations thereof. The abovementioned lactic acid bacteria strains are respectively deposited in China Center for Type Culture Collection (CCTCC) and China General Microbiological Culture Collection Center (CGMCC).

In one embodiment, the vagina-cleaning composition of the present invention comprises a fermentate of lactic acid bacteria, and a physiologically-acceptable excipient, diluent or carrier. The fermentate of lactic acid bacteria is generated by jointly fermenting an isolated lactic acid bacteria strain and Streptococcus thermophilus in a medium containing at least one of milk, powdered milk, and casein. The lactic acid bacteria strain comprises an AP-32 strain of Lactobacillus salivarius subsp. Salicinius (CCTCC NO. M2011127); an F-1 strain of Lactobacillus rhamnosus (CCTCC NO. M2011124); a GL-165 strain of Lactobacillus rhamnosus (CCTCC NO. M2014591); a CP-9 strain of Bifidobacterium animalis subsp. lactis (CCTCC NO. M2014588); a Bf-688 strain of Bifidobacterium bifidum (CGMCC NO. 17953); a Bv-889 strain of Bifidobacterium breve (CGMCC NO. 16145) or combinations thereof. The abovementioned lactic acid bacteria strains are respectively deposited in China Center for Type Culture Collection (CCTCC) and China General Microbiological Culture Collection Center (CGMCC).

In one embodiment, the present invention also provides a use of a composition containing a fermentate of lactic acid bacteria for inhibiting vaginitis pathogens comprising administering to a subject the composition, wherein the composition containing the fermentate of lactic acid bacteria comprises a fermentate of lactic acid bacteria, and an excipient, diluent or carrier. The fermentate of lactic acid bacteria is generated by jointly fermenting an isolated lactic acid bacteria strain and Streptococcus thermophilus in a medium containing at least one of milk, powdered milk, and casein. The lactic acid bacteria strain comprises an AP-32 strain of Lactobacillus salivarius subsp. Salicinius (CCTCC NO. M2011127); an F-1 strain of Lactobacillus rhamnosus (CCTCC NO. M2011124); a GL-165 strain of Lactobacillus rhamnosus (CCTCC NO. M2014591); a CP-9 strain of Bifidobacterium animalis subsp. lactis (CCTCC NO. M2014588); a Bf-688 strain of Bifidobacterium bifidum (CGMCC NO. 17953); a Bv-889 strain of Bifidobacterium breve (CGMCC NO. 16145) or combinations thereof. The abovementioned lactic acid bacteria strains are respectively deposited in China Center for Type Culture Collection (CCTCC) and China General Microbiological Culture Collection Center (CGMCC).

Below, embodiments are described in detail in cooperation with the attached drawings to make easily understood the objectives, technical contents, characteristics and accomplishments of the present invention.

### BRIEF DESCRIPTION OF THE DRAWINGS

FIG. 1 shows the effects of the fermentates of different lactic acid bacteria strains of the present invention on inhibiting vaginitis pathogens;

FIG. 2 also shows the effects of the fermentates of different lactic acid bacteria strains of the present invention on inhibiting vaginitis pathogens;

FIG. 3 shows the effects of the fermentate of mixed lactic acid bacteria strains of the present invention on inhibiting vaginitis pathogens;

FIG. 4 is a histogram showing the bacteria inhibition rates of the fermentate of mixed lactic acid bacteria strains of the present invention; and

FIG. 5 is a histogram showing the bacteria inhibition rates of the gels containing different concentrations of the fermentate of lactic acid bacteria strains of the present invention.

### DESCRIPTION OF THE PREFERRED EMBODIMENTS

The present invention will be described in detail with embodiments and attached drawings below. However, these embodiments are only to exemplify the present invention but not to limit the scope of the present invention. In addition to the embodiments described in the specification, the present invention also applies to other embodiments. Further, any modification, variation, or substitution, which can be easily made by the persons skilled in that art according to the embodiment of the present invention, is to be also included within the scope of the present invention, which is based on the claims stated below. Although many special details are provided herein to make the readers more fully understand the present invention, the present invention can still be practiced under a condition that these special details are partially or completely omitted. Besides, the elements or steps, which are well known by the persons skilled in the art, are not described herein lest the present invention be limited unnecessarily. Similar or identical elements are denoted with similar or identical symbols in the drawings. It should be noted: the drawings are only to depict the present invention schematically but not to show the real dimensions or quantities of the present invention. Besides, matterless details are not necessarily depicted in the drawings to achieve conciseness of the drawings.

The freeze-dried cultures of the strains of lactic acid bacteria mentioned in the specification are respectively deposited in China Center for Type Culture Collection (CCTCC, Wuhan University, Wuhan, 430072, China) and China General Microbiological Culture Collection Center (CGMCC) of Chinese Academy of Sciences (NO. 1 West Beichen Road, Chaoyang District, Beijing, 100101, China). The details thereof are listed in Table. 1.

TABLE 1
Data of Deposited Strains of Lactic Acid Bacteria

| Strain | Species | Deposition No. | Deposition Date |
|---|---|---|---|
| AP-32 | Lactobacillus salivarius | CCTCC NO.M2011127 | 2011 Apr. 10 |
| | subsp. salicinius | | |
| F-1 | Lactobacillus rhamnosus | CCTCC NO.M2011124 | 2011 Apr. 10 |
| GL-165 | Lactobacillus rhamnosus | CCTCC NO.M2014591 | 2014 Nov. 24 |
| CP-9 | Bifidobacterium animalis | CCTCC NO.M2014588 | 2014 Nov. 24 |
| | subsp. lactis | | |
| Bf-688 | Bifidobacterium bifidum | CGMCC NO.17953 | 2019 Jun. 18 |
| Bv-889 | Bifidobacterium breve | CGMCC NO.16145 | 2018 Jul. 23 |

It is found: the fermentates of the lactic acid bacteria strains listed in Table. 1 have the effect of inhibiting vaginitis pathogens. Therefore, the fermentates of the lactic acid bacteria strains listed in Table. 1 may be applied to inhibiting vaginitis pathogens.

In one embodiment, the vaginitis pathogen-inhibiting composition of the present invention comprises a fermentate of lactic acid bacteria, and an excipient, diluent or carrier. The fermentate of lactic acid bacteria is generated by jointly fermenting an isolated lactic acid bacteria strain and Streptococcus thermophilus in a medium containing at least one of milk, powdered milk, and casein. The lactic acid bacteria strain is at least one lactic acid bacteria strain selected from a group including an AP-32 strain of Lactobacillus salivarius subsp. Salicinius (CCTCC NO. M2011127); an F-1 strain of Lactobacillus rhamnosus (CCTCC NO. M2011124); a GL-165 strain of Lactobacillus rhamnosus (CCTCC NO. M2014591); a CP-9 strain of Bifidobacterium animalis subsp. lactis (CCTCC NO. M2014588); a Bf-688 strain of Bifidobacterium bifidum (CGMCC NO. 17953); a Bv-889 strain of Bifidobacterium breve (CGMCC NO. 16145) or combinations thereof. The abovementioned lactic acid bacteria strains are respectively deposited in China Center for Type Culture Collection (CCTCC) and China General Microbiological Culture Collection Center (CGMCC).

In one embodiment, the excipient, diluent or carrier is a physiologically-acceptable excipient, diluent or carrier, which enables the composition of the present invention to function as a food composition or a vagina-cleaning composition. In one embodiment, the excipient, diluent or carrier is a pharmaceutically-acceptable excipient, diluent or carrier, which enables the composition of the present invention to function as a pharmaceutical composition.

In the embodiment of the food composition, the physiologically-acceptable excipient, diluent or carrier is a food. The food may be but is not limited to be dairy food, tea, coffee, a functional beverage, or a combination thereof. The dairy food may be fermented milk, yoghurt, cheese, or powdered milk.

In the embodiment of the pharmaceutical composition, the pharmaceutical composition may be in form of an oral dosage or a topical dosage. The oral dosage may be a tablet, a capsule, a solution, or a powder. The topical dosage may be a powder, an ointment, a spray, a gel, a solution, a pulvis, or a cream.

In the embodiment of the vagina-cleaning composition, the vagina-cleaning composition may be in form of a tablet, a capsule, a powder, an ointment, a spray, a gel, a solution, a pulvis, a cream, or a lotion.

In one embodiment, the fermentate generated by the lactic acid bacteria strain of the present invention may be a fermented liquid or a dried powder of the fermented liquid. In one embodiment, the fermented liquid or the dried powder of the fermented liquid contains inactivated strains. In one embodiment, bacteria bodies are removed from the fermented liquid or the dried powder of the fermented liquid. In one embodiment, the fermented liquid may be a supernatant of a fermented liquid or a fermented whey. In one embodiment, the fermentate of the lactic acid bacteria of the vaginitis pathogen-inhibiting composition contains over 0.4% the dried powder of the fermentate generated by the lactic acid bacteria. In one embodiment, the fermentate of the lactic acid bacteria of the vaginitis pathogen-inhibiting composition contains over 2% the solution of the fermentate generated by the lactic acid bacteria.

In one embodiment, the vaginitis pathogen-inhibiting composition of the present invention further comprises an anti-inflammatory agent or a preservative. The anti-inflammatory agent or preservative may be a first composite, a second composite, a third composite, menthol, tea tree essential oil, aloe extract, plectranthus amboinicus extract, silver nanoparticles, or a combination thereof. The first composite includes dipropylene glycol, hydroxyacetophenone, caprylyl glycol, and dipotassium glycyrrhizinate. The first composite is called the activonol-M in the related field. The second composite includes Glycine Soja Seed Extract, Houttuynia Cordata Extract, Scutellaria Baicalensis Root Extract, Melia Azadirachta Leaf Extract, Rehmannia Chinensis Root Extract, Salix Alba Bark Extract, Phellodendron Amurense Bark Extract, o-Cymen-5-Ol, lactobacilli, Pear Juice Ferment Filtrate, Glyceryl Caprylate, Ethylhexylglycerin, PEG-60 Hydrogenated Castor Oil, Glycolic Acid, Butylene Glycol, and water. The second composite is called the acnebusters in the related field. The third composite includes Zanthoxylum Piperitum Fruit Extract, Pulsatilla Koreana Root Extract, and Usnea Barbata Extract. The third composite is called the EURO-Napre in the related field. In one embodiment, the vaginitis pathogen-inhibiting composition of the present invention contains 0.1-8% the anti-inflammatory agent or preservative.

In one embodiment, the vaginitis pathogen-inhibiting composition of the present invention further comprises a gel agent. The gel agent may be a natural dextran glue, carbomer, or a combination thereof. In one embodiment, the vaginitis pathogen-inhibiting composition of the present invention contains 0.2-5% the gel agent.

In one embodiment, the vaginitis pathogen-inhibiting composition of the present invention further comprises a functional component. The functional component may be sodium hyaluronate, an anti-allergy complex, allantoin, or a combination thereof. The anti-allergy complex includes deep ocean water and squash seed extract. The anti-allergy complex is called the ocaline XP in the related field. In one embodiment, the vaginitis pathogen-inhibiting composition of the present invention contains 0.005-5% the functional component.

In one embodiment, the excipient, diluent or carrier comprises sodium hydroxide, triethanolamine, glycerol, or a combination thereof, wherein the excipient, diluent or carrier contains 0.05-10% sodium hydroxide, triethanolamine, glycerol or a combination thereof, and pure water is the balance.

### Embodiment I: Morphologies and General Properties of the Lactic Acid Bacteria Strains of the Present Invention

The taxonomic characteristics of the strains are identified with the 16S rDNA sequencing analysis and the API bacterial identification system. The morphologies and general properties of the lactic acid bacteria strains of the present invention are listed in Table. 2.

| Strain | Morphology and characteristics |
|---|---|
| AP-32 strain of | 1. They are gram-positive bacilli, unlikely to generate spores, free |
| Lactobacillus salivarius | of catalase, oxidase and motility, able to grow in aerobic and |
| subsp. Salicinius | anaerobic environments, most suitable to grow at a temperature |
| | of 37 ± 1° C. They belong to a facultative heterofermentative |
| | strains and do not generate gas in glucose metabolism. |
| | 2. While AP-32 is cultured in the MRS medium, the colony |
| | thereof has a shape of a solid circle and a color of white, the |
| | bodies of the bacteria each have a shape of a short rod, and the |
| | ends of the body are circular-shaped. They often appear in |
| | single bodies. |
| F-1 strain of | 1. While F-1 is cultured in the MRS medium, the bodies of the |
| Lactobacillus | bacteria each have a shape of a short rod or a longer rod, and the |
| rhamnosus | ends of the body are square-shaped. They often appear in single |
| | bodies, in pairs, or in short chains. |
| | 2. They are gram-positive bacilli, unlikely to generate spores, free |
| | of catalase, oxidase and motility, able to grow in aerobic and |
| | anaerobic environments, most suitable to grow at a temperature |
| | of 37 ± 1° C. They belong to a facultative heterofermentative |
| | strains and do not generate gas in glucose metabolism. |
| GL-165 strain of | 1. While GL-165 is cultured in the MRS medium, the colony |
| Lactobacillus | thereof has a shape of a solid circle and a color of white, the |
| rhamnosus | bodies of the bacteria each have a shape of a short rod, and the |
| | ends of the body are oval-shaped. They often appear in single |
| | bodies or short chains. |
| | 2. They are gram-positive bacilli, unlikely to generate spores, free |
| | of catalase, oxidase and motility, able to grow in aerobic and |
| | anaerobic environments, most suitable to grow at a temperature |
| | of 37 ± 1° C. They belong to a facultative heterofermentative |
| | strains and do not generate gas in glucose metabolism. |
| CP-9 strain of | 1. They are anaerobic bacteria. They are gram-positive bacilli, |
| Bifidobacterium | unlikely to generate spores, free of catalase, oxidase and |
| animalis subsp. lactis | motility, able to grow in absolutely anaerobic environments, |
| | most suitable to grow at a temperature of 37 ± 1° C. They belong |
| | to a facultative heterofermentative strains and do not generate |
| | gas in glucose metabolism. |
| | 2. While CP-9 is cultured in the MRS medium, the colony thereof |
| | has a shape of solid circles and a color of white. The bacterium |
| | body has a middle-size or longer rod-like shape, and two ends |
| | thereof sometimes have Y (or V)-shaped branches. |
| Bf-688 strain of | 1. They are anaerobic bacteria. They are gram-positive bacilli, |
| Bifidobacterium | unlikely to generate spores, free of catalase, oxidase and |
| bifidum | motility, able to grow in absolutely anaerobic environments, |
| | most suitable to grow at a temperature of 37 ± 1° C. They belong |
| | to a facultative heterofermentative strains and do not generate |
| | gas in glucose metabolism. |
| | 2. While Bf-688 is cultured in the MRS medium, the colony |
| | thereof has a shape of solid circles and a color of white. The |
| | bacterium body has a middle-size or longer rod-like shape, and |
| | two ends thereof sometimes have Y (or V)-shaped branches. |
| Bv-889 strain of | 1. They are anaerobic bacteria. They are gram-positive bacilli, |
| Bifidobacterium breve | unlikely to generate spores, free of catalase, oxidase and |
| | motility, able to grow in absolutely anaerobic environments, |
| | most suitable to grow at a temperature of 37 ± 1° C. They belong |
| | to a facultative heterofermentative strains and do not generate |
| | gas in glucose metabolism. |
| | 2. While Bv-889 is cultured in the MRS medium, the colony |
| | thereof has a shape of solid circles and a color of white. The |
| | bacterium body has a middle-size or shorter rod-like shape, and |
| | two ends thereof sometimes have Y (or V)-shaped branches. |

### Embodiment II: Collecting the Fermentate of Lactic Acid Bacteria of the Present Invention

The fermentate of the lactic acid bacteria of the present invention is generated by jointly fermenting Streptococcus thermophilus and an isolated lactic acid bacteria strain, which is selected from a group including an AP-32 strain of Lactobacillus salivarius subsp. Salicinius, an F-1 strain of Lactobacillus rhamnosus, a GL-165 strain of Lactobacillus rhamnosus, a CP-9 strain of Bifidobacterium animalis subsp. Lactis, a Bf-688 strain of Bifidobacterium bifidum, and a Bv-889 strain of Bifidobacterium breve, in a medium containing at least one of milk, powdered milk, and casein. The fermentate is centrifuged to obtain a fermentate liquid. It should be explained: Streptococcus thermophilus is used to assist in fermentation. Therefore, the strain of Streptococcus thermophilus is not particularly limited herein. According to requirement, the fermentate liquid may be further dried to form a fermentate powder.

### Embodiment III: Analyzing the Effects of the Lactic Acid Bacteria of the Present Invention on Inhibiting Vaginitis Pathogens

The evaluation of the effect of the lactic acid bacteria on inhibiting vaginitis pathogens is according to the method for evaluating the anti-bacteria capability in a paper, which is published by Strus in 2005 on Infectious Diseases in Obstetrics and Gynecology, 13(2), 69-75, Canada. Firstly, smear the strains of the lactic acid strains on solid-state mediums by a smear width of 2 cm, and the strains include the AP-32 strain of Lactobacillus salivarius subsp. Salicinius, the F-1 strain of Lactobacillus rhamnosus, the GL-165 strain of Lactobacillus rhamnosus, the CP-9 strain of Bifidobacterium animalis subsp. Lactis, the Bf-688 strain of Bifidobacterium bifidum, and the Bv-889 strain of Bifidobacterium breve, and these strains have been activated to the third generation. An SY-66 strain of Streptococcus thermophiles is used as the negative control group, wherein the lactic acid bacteria are not smeared on the control group. The mediums are respectively cultured according to the corresponding conditions of culturing the lactic acid bacteria for 2 days. Next, fill 14 ml of second mediums for culturing vaginitis pathogens into the abovementioned solid-state mediums, and let the second mediums solidify. Next, evenly smear the liquids of vaginitis pathogens onto the second mediums. The vaginitis pathogens include Candida albicans, Staphylococcus aureus, Escherichia coli, Methicillin-resistant Staphylococcus aureus (MRSA), Group B Streptococcus agalactiae (GBS), and Escherichia coli-ESBL. The second mediums on which the vaginitis pathogens are smeared are respectively cultured according to the corresponding conditions of culturing the pathogens for corresponding time intervals. Next, take out the culture dishes, and measure the pathogen-inhibiting widths to evaluate the anti-bacteria capabilities of the fermentates of the lactic acid bacteria strains. The higher the score, the better the anti-bacteria capability. The standard for evaluating the anti-bacteria capability is shown in Table. 3.

TABLE 3
The standard for evaluating the anti-bacteria capability

| | Pathogen-inhibiting | |
|---|---|---|
| Symbol | width (cm) | Score |
| (−) | 0 | 0 |
| (+/−) | <2 | 1 |
| (++) | <3 | 2 |
| (+++) | <4 | 3 |
| (++++) | <5 | 4 |
| (+++++) | <9 | 5 |
| (++++++) | 9 | 6 |

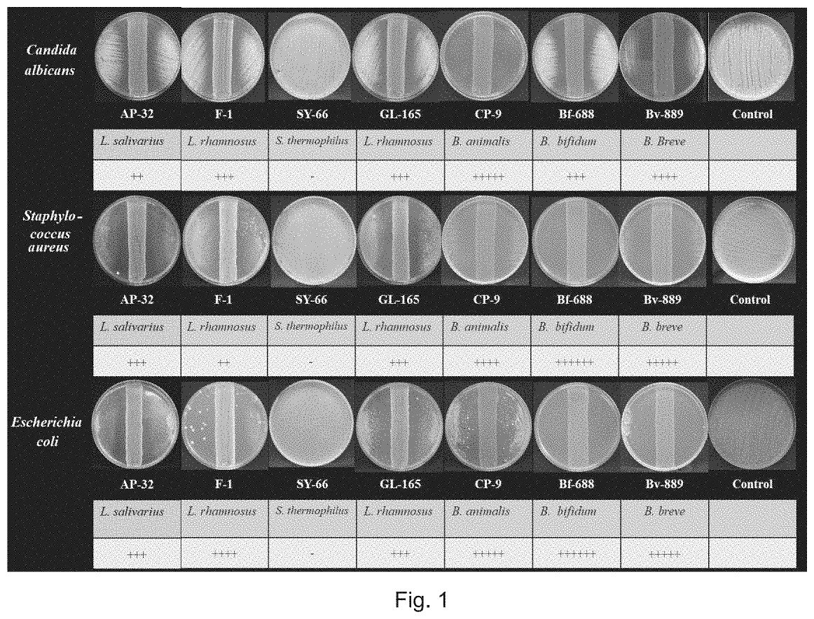

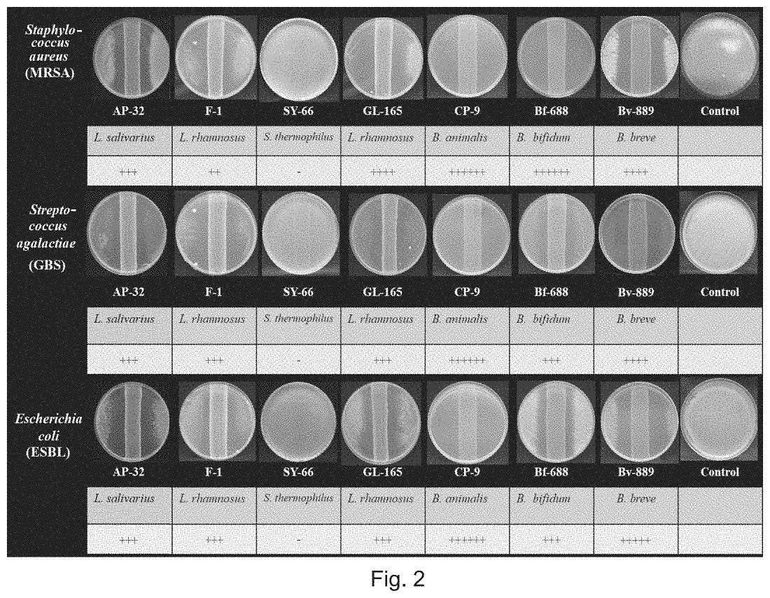

The results of analyzing the effects of the fermentates of the lactic acid bacteria strains of the present invention on inhibiting vaginitis pathogens are shown in FIG. 1 and FIG. 2. From FIG. 1 and FIG. 2, it is learned: the fermentates of the lactic acid bacteria strains of the present invention have activities to inhibit vaginitis pathogens.

### Embodiment IV: Analyzing the Effects of the Fermentates of the Lactic Acid Bacteria of the Present Invention on Inhibiting Vaginitis Pathogens (1)

Below is described a method for evaluating the effects of the fermentates of the lactic acid bacteria on inhibiting vaginitis pathogens. In one embodiment, jointly ferment Streptococcus thermophilus and an isolated lactic acid bacteria strain, which is selected from a group including an AP-32 strain of Lactobacillus salivarius subsp. Salicinius, an F-1 strain of Lactobacillus rhamnosus, a GL-165 strain of Lactobacillus rhamnosus, a CP-9 strain of Bifidobacterium animalis subsp. Lactis, a Bf-688 strain of Bifidobacterium bifidum, and a Bv-889 strain of Bifidobacterium breve, in a medium containing at least one of milk, powdered milk, and casein. Next, the fermentate is centrifuged to obtain the upper layer liquid of the fermentate.

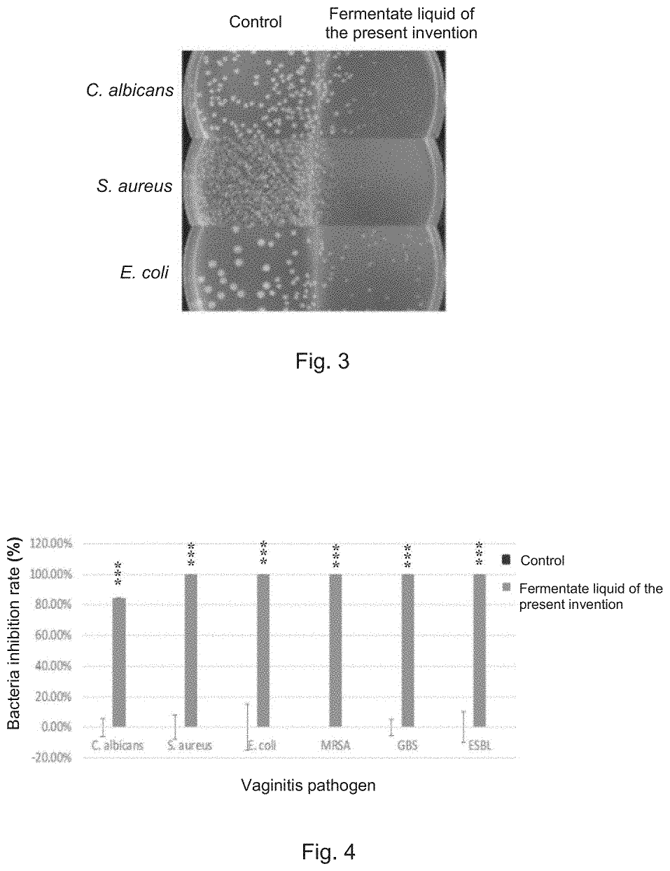

Use the fermentate liquid of the lactic acid bacteria of the present invention as the solvent of the powdered medium for culturing vaginitis pathogens. Use a supernatant, which does not contain the fermentate of the lactic acid bacteria, to prepare a medium as the control group. The mediums are sterilized, and then the mediums are respectively cast into culture dishes to fill a half area of each culture dish. After the mediums solidify, pour the medium containing the control group into the culture dishes and fill another half area of each culture dish. Next, smear the liquids containing the vaginitis pathogens (including Candida albicans, Staphylococcus aureus, and Escherichia coli) onto the abovementioned mediums. Next, cultivate the vaginitis pathogens according to corresponding conditions for corresponding time intervals. Next, take out the culture dishes, and compare the anti-bacteria effects of the fermentate liquid of the lactic acid bacteria of the present invention with the anti-bacteria effect of the control group. The test results are shown in FIG. 3. From FIG. 3, it is learned: the vaginitis pathogen-inhibiting effects of the fermentate liquid of the lactic acid bacteria of the present invention are better than the vaginitis pathogen-inhibiting effect of the control group.

### Embodiment V: Analyzing the Effects of the Fermentates of the Lactic Acid Bacteria of the Present Invention on Inhibiting Vaginitis Pathogens (2)

Below is described another method for evaluating the effects of the fermentates of the lactic acid bacteria on inhibiting vaginitis pathogens. Fill a fixed amount (4.9 mL) of liquid containing the fermentate liquid of the lactic acid bacteria of the present invention into culture tubes. Use a liquid medium, which does not contain the fermentate of the lactic acid bacteria, as the control group. Next, add 0.1 mL bacterial liquids respectively containing activated vaginitis pathogens to the culture tubes (including Candida albicans, Staphylococcus aureus, Escherichia coli, Methicillin-resistant Staphylococcus aureus (MRSA), Group B Streptococcus agalactiae (GBS), and Escherichia coli (ESBL). Next, cultivate the vaginitis pathogens according to corresponding conditions for corresponding time intervals. Next, dilute the medium containing the control group and the mediums containing the fermentate liquid, in which where the vaginitis pathogens are cultivated. Next, smear the diluted medium of the control group and the experimental groups onto the culture dishes carrying the solid-state mediums for cultivating the vaginitis pathogens. Next, cultivate the culture dishes according to the corresponding conditions of culturing the vaginitis pathogens for corresponding culture time intervals. Next, take out the culture dishes, count the number of the colonies of the pathogens, and calculate the bacteria inhibition rates. The bacteria inhibition rate is calculated according to the following equation:

Bacteria inhibition rate (%)=(1-colony number of experimental group/colony number of control group)×100%

The test results are shown in FIG. 4, wherein *** means p<0.001, indicating that the difference is very significant in statistics. From FIG. 4, it is learned: the fermentate liquid of the lactic acid bacteria of the present invention has a significant inhibiting effect on vaginitis pathogens, and the inhibition rates of the fermentate liquid of the present invention to the vaginitis pathogens are all over 80%.

### Embodiment VI: Analyzing the Effects of the Gels of the Fermentates of the Lactic Acid Bacteria of the Present Invention on Inhibiting Vaginitis Pathogens

Below is described a method for evaluating the effects of the gels of the fermentates of the lactic acid bacteria on inhibiting vaginitis pathogens. Firstly, take the following components by appropriate proportions: a gel agent (such as a natural dextran glue or carbomer), a functional component (such as sodium hyaluronate, the ocaline XP, allantoin, or a combination thereof), and 50% water; then mix and agitate them to completely dissolve. Next, add the following components to the solution: the fermentate liquid of the lactic acid bacteria of the present invention (by a proportion of 2%, 5%, or 10%), and an anti-inflammatory agent or preservative (such as the activonol-M, the acnebusters, the EURO-Napre, menthol, tea tree essential oil, aloe extract, plectranthus amboinicus extract, silver nanoparticles, or a combination thereof); mix and agitate them evenly. Next, add an excipient (such as sodium hydroxide, triethanolamine, glycerol, or a combination thereof) to the solution; modify the pH value thereof to 4.5-6.5; add pure water to be the balance of the excipient; agitate them evenly. A first control group does not contain the fermentate liquid of the lactic acid bacteria of the present invention, the functional component and the anti-inflammatory agent/preservative but merely contains the gel agent. A second control group does not contain the fermentate liquid of the lactic acid bacteria of the present invention but merely contains the gel agent and the anti-inflammatory agent/preservative. Next, respectively add 0.1 ml activated vaginitis pathogens at a concentration of 1×10⁵-9×10⁵ CFU/ml (Candida albicans, Staphylococcus aureus, and Escherichia coli) to 2.5 g the gels of each group; mix them evenly; then place them for 20 minutes. Next, respectively add 22.5 ml water to the 2.5 g gels to dilute gels 10 times. Next, take 0.1 ml of the diluted gels to smear them onto the culture dishes in an appropriate sequence, and cultivate them at a temperature of 37° C. Then, calculate the bacteria inhibition rates according to the abovementioned method.

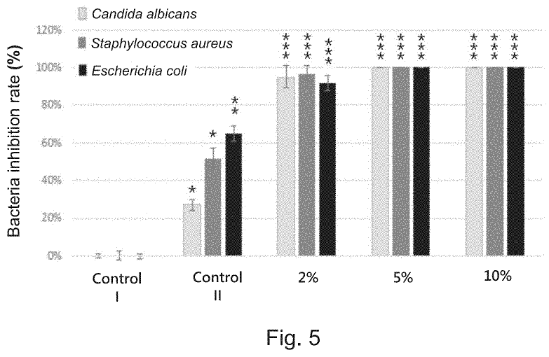

FIG. 5 shows the results of analyzing the effects of the gels of the fermentates of the lactic acid bacteria of the present invention on inhibiting vaginitis pathogens, wherein *** means p<0.001, ** means p<0.01, both indicating that the difference is very significant in statistics; * means p<0.05, indicating that the difference is significant in statistics. It is learned from FIG. 5: although the bacteria-inhibiting effect of the second control group, which contains merely the anti-inflammatory agent/preservative, is statistically significant with respect to the first control group, the bacteria inhibition rates of the second control group to the tested vaginitis pathogens are all lower than 75%. The gels, which respectively contain 2%, 5% and 10% of the fermentate liquid of the lactic acid bacteria of the present invention, all have statistically significant bacteria-inhibiting effects, and the bacteria inhibition rates thereof are all over 90%. Especially, the gels, which respectively contain 5% and 10% of the fermentate liquid of the lactic acid bacteria of the present invention, both have the bacteria inhibition rates of 100% to all the tested vaginitis pathogens.

In conclusion, the present invention proposes a vaginitis pathogen-inhibiting composition, which comprises a fermentate of lactic acid bacteria, especially the fermentate generated by jointly fermenting Streptococcus thermophiles and a lactic acid bacteria strain in a medium containing at least one of milk, powdered milk, and casein, wherein the lactic acid bacteria strain is selected from a group containing an AP-32 strain of Lactobacillus salivarius subsp. Salicinius; an F-1 strain of Lactobacillus rhamnosus; a GL-165 strain of Lactobacillus rhamnosus; a CP-9 strain of Bifidobacterium animalis subsp. lactis; a Bf-688 strain of Bifidobacterium bifidum; a Bv-889 strain of Bifidobacterium breve; and combinations thereof. The fermentate of the lactic acid bacteria strains of the present invention can inhibit growth of vaginitis pathogens and may be in form of a food composition, a pharmaceutical composition, or a vagina-cleaning composition. In preferred embodiments, the vaginitis pathogen-inhibiting composition of the present invention also comprises an anti-inflammatory agent/preservative or a functional component, whereby to further enhance the vaginitis pathogen-inhibiting capability of the present invention.

The embodiments have been described above to demonstrate the technical thoughts and characteristics of the present invention to enable the persons skilled in the art to understand, make, and use the present invention. However, these embodiments are only to exemplify the present invention but not to limit the scope of the present invention. Any equivalent modification or variation according to the spirit of the present invention is to be also included within the scope of the present invention.

Bioresource Deposition

(1) AP-32 Strain
Deposition Number: CCTCC NO: M2011127;
Deposition Date: Apr. 10, 2011;
Deposition Organization: China Center for Type Culture Collection (CCTCC);
Address of Deposition Organization: Wuhan University, Wuhan, 430072, China;
Nomenclature: Lactobacillus salivarius subsp. Salicinius);

(2) F-1 Strain
Deposition Number: CCTCC NO. M2011124;
Deposition Date: Apr. 10, 2011;
Deposition Organization: China Center for Type Culture Collection (CCTCC);
Address of Deposition Organization: Wuhan University, Wuhan, 430072, China;
Nomenclature: Lactobacillus rhamnosus;

(3) GL-165 Strain
Deposition Number: CCTCC NO. M2014591;
Deposition Date: Nov. 24, 2014;
Deposition Organization: China Center for Type Culture Collection (CCTCC);
Address of Deposition Organization: Wuhan University, Wuhan, 430072, China;
Nomenclature: Lactobacillus rhamnosus;

(4) CP-9 Strain
Deposition Number: CCTCC NO. M2014591;
Deposition Date: Nov. 24, 2014;
Deposition Organization: China Center for Type Culture Collection (CCTCC);
Address of Deposition Organization: Wuhan University, Wuhan, 430072, China;
Nomenclature: Bifidobacterium animalis subsp. lactis;

(5) Bf-688 Strain
Deposition Number: CGMCC NO. 17953;
Deposition Date: Jun. 18, 2019;
Deposition Organization: China General Microbiological Culture Collection Center (CGMCC);
Address of Deposition Organization: Institute of Microbiology, Chinese Academy of Sciences, NO. 1 Beichen West Road, Chaoyang District, Beijing, 100101, China;
Nomenclature: Bifidobacterium bifidum;

(6) Bv-889 Strain
Deposition Number: CGMCC NO. 16145;
Deposition Date: Jul. 23, 2018;
Deposition Organization: China General Microbiological Culture Collection Center (CGMCC);
Address of Deposition Organization: Institute of Microbiology, Chinese Academy of Sciences, NO. 1 Beichen West Road, Chaoyang District, Beijing, 100101, China;
Nomenclature: Bifidobacterium breve.

What is claimed is:

1. A vaginitis pathogen-inhibiting composition comprising:
a fermentate of lactic acid bacteria, wherein the fermentate is generated by fermenting an isolated lactic acid bacteria strain and Streptococcus thermophilus jointly in a medium containing at least one of milk, powdered milk, and casein, and wherein the lactic acid bacteria strain comprises an AP-32 strain of Lactobacillus salivarius subsp. Salicinius (CCTCC NO. M2011127); an F-1 strain of Lactobacillus rhamnosus (CCTCC NO. M2011124); a GL-165 strain of Lactobacillus rhamnosus (CCTCC NO. M2014591); a CP-9 strain of Bifidobacterium animalis subsp. lactis (CCTCC NO. M2014588); a Bf-688 strain of Bifidobacterium bifidum (CGMCC NO. 17953); a Bv-889 strain of Bifidobacterium breve (CGMCC NO. 16145) or combinations thereof, and wherein the abovementioned lactic acid bacteria strains are respectively deposited in China Center for Type Culture Collection (CCTCC) and China General Microbiological Culture Collection Center (CGMCC); and
an excipient, diluent or carrier.

2. The vaginitis pathogen-inhibiting composition according to claim 1, wherein the fermentate of lactic acid bacteria comprises inactivated strains, or a supernatant of a fermented liquid, a fermented whey or a dried powder thereof in which bacteria are removed.

3. The vaginitis pathogen-inhibiting composition according to claim 1, which contains 0.4-10% the fermentate of lactic acid bacteria.

4. The vaginitis pathogen-inhibiting composition according to claim 1, wherein the excipient, diluent or carrier is a food.

5. The vaginitis pathogen-inhibiting composition according to claim 4, wherein the food is fermented milk, yoghurt, cheese, powdered milk, tea, coffee, a functional beverage, or a combination thereof.

6. The vaginitis pathogen-inhibiting composition according to claim 1, wherein the excipient, diluent or carrier is a pharmaceutically-acceptable excipient, diluent or carrier.

7. The vaginitis pathogen-inhibiting composition according to claim 6, which is in form of an oral dosage or a topical dosage, wherein the oral dosage is in form of a tablet, a capsule, a solution, or a powder, and wherein the topical dosage is in form of a powder, an ointment, a spray, a gel, a solution, a pulvis, or a cream.

8. The vaginitis pathogen-inhibiting composition according to claim 6 further comprising an anti-inflammatory agent or a preservative.

9. The vaginitis pathogen-inhibiting composition according to claim 8, wherein the anti-inflammatory agent or the preservative includes a first composite, a second composite, a third composite, menthol, tea tree essential oil, aloe extract, plectranthus amboinicus extract, silver nanoparticles, or a combination thereof, and wherein a concentration of the anti-inflammatory agent or the preservative is 0.1-8%.

10. The vaginitis pathogen-inhibiting composition according to claim 6 further comprising a gel agent.

11. The vaginitis pathogen-inhibiting composition according to claim 10, wherein the gel agent includes a natural dextran glue, carbomer, or a combination thereof, and wherein a concentration of the gel agent is 0.2-5%.

12. The vaginitis pathogen-inhibiting composition according to claim 6 further comprising a functional component.

13. The vaginitis pathogen-inhibiting composition according to claim 12, wherein the functional component includes sodium hyaluronate, an anti-allergy complex, allantoin, or a combination thereof, and wherein a concentration of the functional component is 0.005-5%, and wherein the anti-allergy complex includes deep ocean water and squash seed extract.

14. The vaginitis pathogen-inhibiting composition according to claim 1, wherein, the excipient, diluent or carrier comprises sodium hydroxide, triethanolamine, glycerol, or a combination thereof, and wherein a concentration of the excipient, diluent or carrier is 0.05-10%, and pure water is the balance.

15. A vagina-cleaning composition comprises:
a fermentate of lactic acid bacteria, wherein the fermentate is generated by fermenting an isolated lactic acid bacteria strain and Streptococcus thermophilus jointly in a medium containing at least one of milk, powdered milk, and casein, and wherein the lactic acid bacteria strain comprises an AP-32 strain of Lactobacillus salivarius subsp. Salicinius (CCTCC NO. M2011127); an F-1 strain of Lactobacillus rhamnosus (CCTCC NO. M2011124); a GL-165 strain of Lactobacillus rhamnosus (CCTCC NO. M2014591); a CP-9 strain of Bifidobacterium animalis subsp. lactis (CCTCC NO. M2014588); a Bf-688 strain of Bifidobacterium bifidum (CGMCC NO. 17953); a Bv-889 strain of Bifidobacterium breve (CGMCC NO. 16145) or combinations thereof, and wherein the abovementioned lactic acid bacteria strains are respectively deposited in China Center for Type Culture Collection (CCTCC) and China General Microbiological Culture Collection Center (CGMCC); and
a physiologically-acceptable excipient, diluent, or carrier.

16. The vagina-cleaning composition according to claim 15, wherein the fermentate of lactic acid bacteria comprises inactivated strains, or a supernatant of a fermented liquid, a fermented whey or a dried powder thereof in which bacteria are removed.

17. The vagina-cleaning composition according to claim 15, wherein the excipient, diluent or carrier is in form of a tablet, a capsule, a powder, an ointment, a spray, a gel, a solution, a pulvis, a cream, or a lotion.

18. The vagina-cleaning composition according to claim 15, which contains 0.4-10% the fermentate of lactic acid bacteria.

19. A use of a composition containing a fermentate of lactic acid bacteria for inhibiting vaginitis pathogens comprising administering to a subject the composition, wherein the composition containing the fermentate of lactic acid bacteria comprises:
a fermentate of lactic acid bacteria, wherein the fermentate is generated by fermenting an isolated lactic acid bacteria strain and Streptococcus thermophilus jointly in a medium containing at least one of milk, powdered milk, and casein, and wherein the lactic acid bacteria strain comprises an AP-32 strain of Lactobacillus salivarius subsp. Salicinius (CCTCC NO. M2011127); an F-1 strain of Lactobacillus rhamnosus (CCTCC NO. M2011124); a GL-165 strain of Lactobacillus rhamnosus (CCTCC NO. M2014591); a CP-9 strain of Bifidobacterium animalis subsp. lactis (CCTCC NO. M2014588); a Bf-688 strain of Bifidobacterium bifidum (CGMCC NO. 17953); a Bv-889 strain of Bifidobacterium breve (CGMCC NO. 16145) or combinations thereof, and wherein the abovementioned lactic acid bacteria strains are respectively deposited in China Center for Type Culture Collection (CCTCC) and China General Microbiological Culture Collection Center (CGMCC); and
an excipient, diluent or carrier.

20. The use of the composition containing the fermentate of lactic acid bacteria for inhibiting vaginitis pathogens according to claim 19, wherein the fermentate of lactic acid bacteria comprises inactivated strains, or a supernatant of a fermented liquid, a fermented whey or a dried powder thereof in which bacteria are removed.

21. The use of the composition containing the fermentate of lactic acid bacteria for inhibiting vaginitis pathogens according to claim 19, wherein a concentration of the fermentate of lactic acid bacteria is 0.4-10%.

22. The use of the composition containing the fermentate of lactic acid bacteria for inhibiting vaginitis pathogens according to claim 19, wherein the composition containing a fermentate of lactic acid bacteria further comprises an anti-inflammatory agent or a preservative.

23. The use of the composition containing the fermentate of lactic acid bacteria for inhibiting vaginitis pathogens according to claim 22, wherein the anti-inflammatory agent or the preservative includes a first composite, a second composite, a third composite, menthol, tea tree essential oil, aloe extract, plectranthus amboinicus extract, silver nanoparticles, or a combination thereof, and wherein a concentration of the anti-inflammatory agent or the preservative is 0.1-8%.

24. The use of the composition containing the fermentate of lactic acid bacteria for inhibiting vaginitis pathogens according to claim 19, wherein the composition containing a fermentate of lactic acid bacteria further comprises a gel agent.

25. The use of the composition containing the fermentate of lactic acid bacteria for inhibiting vaginitis pathogens according to claim 24, wherein the gel agent includes a natural dextran glue, carbomer, or a combination thereof, and wherein a concentration of the gel agent is 0.2-5%.

26. The use of the composition containing the fermentate of lactic acid bacteria for inhibiting vaginitis pathogens according to claim 19, wherein the composition containing a fermentate of lactic acid bacteria further comprises a functional component.

27. The use of the composition containing the fermentate of lactic acid bacteria for inhibiting vaginitis pathogens according to claim 26, wherein the functional component includes sodium hyaluronate, an anti-allergy complex, allantoin, or a combination thereof, and wherein a concentration of the functional component is 0.005-5%, and wherein the anti-allergy complex includes deep ocean water and squash seed extract.

28. The use of the composition containing the fermentate of lactic acid bacteria for inhibiting vaginitis pathogens according to claim 19, wherein the excipient, diluent or carrier comprises sodium hydroxide, triethanolamine, glycerol, or a combination thereof, and wherein a concentration of the excipient, diluent or carrier is 0.05-10%, and pure water is the balance.

29. The use of the composition containing the fermentate of lactic acid bacteria for inhibiting vaginitis pathogens according to claim 19, wherein the composition containing the fermentate of lactic acid bacteria is a food composition, a pharmaceutical composition, or a vagina-cleaning composition.
